(19) United States
(12) Patent Application Publication
(10) Pub. No.: US 2022/0098446 A1
(43) Pub. Date: Mar. 31, 2022

(54) BIO-BASED AND COMPOSTABLE HOT MELT ADHESIVE COMPOSITIONS AND ARTICLES INCLUDING THE SAME

(71) Applicant: H.B. Fuller Company, St. Paul, MN (US)

(72) Inventors: Thomas F. Kauffman, Woodbury, MN (US); Adam M. Pederson, Blaine, MN (US); Kevin P. Davis, Woodbury, MN (US)

(21) Appl. No.: 17/449,371

(22) Filed: Sep. 29, 2021

Related U.S. Application Data
(60) Provisional application No. 63/106,590, filed on Oct. 28, 2020.
(60) Provisional application No. 63/084,949, filed on Sep. 29, 2020.

(51) Int. Cl. C09J 7/35; C09J 11/08; C09J 167/04; A61L 15/58; A61L 15/22

(52) U.S. Cl. CPC C09J 7/35; C09J 11/08; C09J 167/04; A61L 15/585; A61L 15/225; C09J 2433/00; C09J 2301/304; C09J 2301/408; C09J 2467/00; C09J 2491/00; C09J 2475/00; C09J 2203/334

(57) ABSTRACT

The invention includes a hot melt adhesive composition including from 15% by weight to 60% by weight of polyester polymer selected from a group consisting of poly lactic acid based polymer (PLA) and polyhydroxy alkanoate (PHA) based polymer and greater than 30% by weight to 85% by weight, greater than 40% by weight to 85% by weight, or even greater than 50% by weight to 85% by weight of a polyester plasticizer having a weight average molecular weight (M_{w}) as tested by SEC of no greater than 10,000 Da.

This application claims the benefit of U.S. Provisional Patent Application No. 63/084,949 filed on Sep. 29, 2020 and U.S. Provisional Patent Application No. 63/106,590 filed on Oct. 28, 2020, which are incorporated herein.

### BACKGROUND

This application is directed to bio-based and compostable hot melt adhesive compositions that can have pressure sensitive adhesive properties.

Hot melt adhesive compositions are commonly based on petroleum derived polymers. Properties of hot melt adhesives can be adjusted by addition of various materials including tackifying agents (e.g. resins), plasticizers (e.g. oil), and waxes. Many of these materials are also petroleum derived.

It would be useful if a hot melt adhesive composition could be formulated primarily or even entirely with bio-based raw materials. It would further be desirable if a portion, or even most of the hot melt adhesive composition could be compostable.

### SUMMARY

In one aspect, the invention features a hot melt adhesive composition including from 15% by weight to 60% by weight of a polyester polymer selected from a group consisting of poly lactic acid based polymer, polyhydroxy alkanoate based polymer, and blends thereof and, from greater than 40% by weight to 85% by weight of a polyester plasticizer having an oxygenated carbon to total carbon ratio of at least 0.40 and a weight average molecular weight (M_{w}) as tested by Size Exclusion Chromatography (SEC) of no greater than 10,000 daltons (Da).

In a different aspect, the invention features a hot melt adhesive composition including from 15% by weight to 55% by weight of a polyester polymer selected from a group consisting of poly lactic acid based polymer and polyhydroxy alkanoate based polymer, and blends thereof and from 35% by weight to 85% by weight of a polyester plasticizer having a weight average molecular weight (M_{w}) as tested by SEC of no greater than 10,000 Da.

In one embodiment, the hot melt adhesive composition has a loop tack of from 50 gf to 2000 gf. In a different embodiment, at least 50% by weight, at least 70% by weight, or even at least 90% by weight of the hot melt adhesive composition includes components that are compostable. In another embodiment, at least 70% by weight, or even at least 90% by weight of the hot melt adhesive composition includes components that are bio-based.

In one embodiment, the hot melt adhesive composition includes a second plasticizer. In another embodiment, the hot melt adhesive composition includes up to 10% of a wax, or up to 30% by weight of a tackifying agent. In another embodiment, the tackifying agent is bio-based.

In one embodiment, the polyester plasticizer is compostable. In another embodiment, the hot melt adhesive composition includes from 43% by weight to 85% by weight, or even from 50% by weight to 85% by weight of the polyester plasticizer. In a different embodiment, the polyester plasticizer has a weight average molecular weight (M_{w}) as tested by SEC of from 2000 Da to 6000 Da. In another embodiment, the polyester plasticizer is derived from monomers selected from the group consisting of ethylene glycol, 1,2 propanediol, isosorbide, succinic acid and combinations thereof.

In one embodiment, the polyester polymer is a poly lactic acid based polymer that is amorphous. In a different embodiment, the polyester polymer has a weight average molecular weight (M_{w}) as tested by SEC of from 40,000 Da to 300,000 Da, or even from 50,000 Da to 150,000 Da.

In one aspect, the invention features a hot melt adhesive composition including from 15% by weight to 60% by weight of polyester polymer selected from a group consisting of poly lactic acid based polymer and polyhydroxy alkanoate based polymer, and blends thereof and from greater than 50% by weight to 85% by weight of a polyester plasticizer having a weight average molecular weight (M_{w}) as tested by SEC of no greater than 10,000 Da.

In one embodiment, the invention features a tape or label comprising the inventive hot melt adhesive composition. In a different embodiment, the invention features a disposable absorbent article comprising inventive hot melt composition.

Applicants have discovered hot melt adhesive compositions that can include bio-based and compostable content and can further unexpectedly be formulated with greater than 35% by weight, or even greater than 40% by weight of a polyester plasticizer resulting in hot melt adhesive compositions with improved tack and peel properties.

Other features and advantages will be apparent from the following description of the preferred embodiments and from the claims.

### DETAILED DESCRIPTION

### Definitions

“Renewable resource” is used herein to refer to a resource that is produced by a natural process at a rate comparable to its rate of consumption. The resource can be replenished naturally or by engineered agricultural techniques. Examples of renewable resources include but are not limited to plants (e.g., sugar cane, beets, corn, potatoes, citrus fruit (e.g. oranges), woody plants, cellulosic waste, etc.), animals, fish, bacteria, fungi, and forestry products (e.g. pine and spruce trees). These resources can be naturally occurring, hybrids, or genetically engineered organisms. Natural resources such as crude oil, coal and natural gas are not considered renewable as they are derived from materials that will run out or will not be replenished for thousands or even millions of years.

“Bio-based” is used herein to refer to a component of the hot melt adhesive that is produced or is derived from at least 50% by weight of a renewable resource. Bio-based materials are not necessarily compostable.

“Compostable” is used herein to refer to a component of the hot melt adhesive composition that meets all requirements of ASTM D 6400-19 [Disintegration Testing within 84 days of exposure (using ISO 20200), Aerobic Biodegradation at 58±2° C. in up to 180 days (using ASTM 5338-15), and Terrestrial Safety (metals analysis, germination rate, and plant biomass testing following testing protocols defined in the D6400-19 method)].

Compostable materials are not necessarily bio-based.

### Hot Melt Adhesive Composition

The invention includes a hot melt adhesive composition including from 15% by weight to 60% by weight of polyester polymer selected from a group consisting of poly lactic acid (PLA) based polymer and polyhydroxy alkanoate (PHA) based polymer and greater than 40% by weight to 85% by weight, or even greater than 50% by weight to 85% by weight of a polyester plasticizer having an oxygenated carbon to total carbon ratio of at least 0.40 and a weight average molecular weight (M_{w}) as tested by the Molecular Weight by Size Exclusion Chromatography (SEC) test method of no greater than 10,000 Da.

The invention includes a hot melt adhesive composition including from 15% by weight to 55% by weight of polyester polymer selected from a group consisting of poly lactic acid (PLA) based polymer and polyhydroxy alkanoate (PHA) based polymer and from 35% by weight to 60% by weight of a polyester plasticizer having an oxygenated carbon to total carbon ratio of at least 0.40 and a M_{w} as tested by SEC of no greater than 10,000 Da.

The invention also includes a hot melt pressure sensitive adhesive composition including from 15% by weight to 60% by weight of polyester polymer selected from a group consisting of poly lactic acid (PLA) based polymer and polyhydroxy alkanoate (PHA) based polymer having a M_{w} as tested by SEC of from 70,000 Da to 200,000 Da, from 60% by weight to 85% by weight of a polyester plasticizer having an oxygenated carbon to total carbon ratio of at least 0.40 and a M_{w} as tested by SEC of no greater than 10,000 Da, and no greater than 10% by weight of a second polymer.

The invention includes an adhesive composition for disposable absorbent articles including from 15% by weight to 55% by weight of polyester polymer selected from a group consisting of poly lactic acid (PLA) based polymer and polyhydroxy alkanoate (PHA) based polymer having a M_{w} as tested by SEC of from 50,000 Da to 150,000 Da and from 35% by weight to 60% by weight of a polyester plasticizer having an oxygenated carbon to total carbon ratio of at least 0.40 and a M_{w} as tested by SEC of no greater than 10,000 Da.

The invention further includes an adhesive composition for disposable absorbent articles including from 15% by weight to 60% by weight of polyester polymer selected from a group consisting of poly lactic acid (PLA) based polymer and polyhydroxy alkanoate (PHA) based polymer having a M_{w} as tested by SEC of from 50,000 Da to 100,000 Da and from 40% by weight to 60% by weight of a polyester plasticizer having an oxygenated carbon to total carbon ratio of at least 0.40 and a M_{w} as tested by SEC of no greater than 10,000 Da.

The hot melt adhesive compositions of this invention can unexpectedly be formulated with a broad range of polyester plasticizer content i.e. from greater than 35%, or even greater than 40% by weight to 85% by weight, and still result in compatible compositions that do not exude or result in transfer.

This property enables versatility in formulation. For example, the hot melt adhesive composition can be formulated to have a variety of glass transition temperatures (T_{g}) depending on the properties desired in the end use. The hot melt adhesive compositions can have a T_{g} of from −20° C. to 30° C., or even from −10° C. to 20° C.

It should be noted that although hot melt adhesive compositions that form a clear, homogeneous mixture when melted are preferred, marginally compatible blends that exhibit opacity but do not phase separate upon heat aging can also be useful.

The hot melt adhesive composition can include at least 50% by weight, at least 60% by weight, at least 70% by weight, at least 75% by weight, at least 80% by weight, at least 90% by weight, at least 95% by weight, 100% by weight, from 50% by weight to 95% by weight, from 60% by weight to 95% by weight, from 70% by weight to 95% by weight, from 75% by weight to 100% by weight, or even from 80% by weight to 100% of components that are bio-based.

The hot melt adhesive composition can include at least 50% by weight, at least 60% by weight, at least 70% by weight, at least 75% by weight, at least 80% by weight, at least 90% by weight, at least 95% by weight, 100% by weight, from 50% by weight to 95% by weight, from 60% by weight to 95% by weight, from 70% by weight to 95% by weight, from 75% by weight to 100% by weight, or even from 80% by weight to 100% of components that are compostable.

The hot melt adhesive composition can be compostable.

The polyester polymer, the polyester plasticizer, the optional wax, the optional tackifying agent and the optional second polymer can make up at least 85% of the composition, at least 90% of the composition or even at least 95% of the composition.

The hot melt adhesive composition can be a pressure sensitive adhesive. When the hot melt is a pressure sensitive adhesive it can have a Loop Tack of greater than 100 gram-force (gf), from 50 gf to 2000 gf, from 100 gf to 1000 gf, or even from 100 gf to 500 gf.

Polyester Polymer

The hot melt adhesive composition includes a polyester polymer selected from the group consisting of PLA based polymer, PHA based polymer and mixtures thereof. The polyester polymer can be a homopolymer or a copolymer. The polyester polymer can include more than one polyester polymer. The polyester polymer is made from bio-based raw materials and is preferably compostable.

Suitable PLA based polymers can include, for example, homopolymers or copolymers made up of (L-lactide), (D-lactide), and (meso-lactide) monomeric units. While poly(D,L-lactide) and poly(meso-lactide) are essentially amorphous, poly(L-lactide, PLLA) or poly(D-lactide, PDLA) are crystalline in nature and have a crystalline melting point of about 186° C., depending on molecular weight and stereo purity.

Suitable PHA based polymers can include, for example, homopolymers or copolymers made up of hydroxy butyrate, hydroxy valerate, and hydroxy hexanoate monomeric units.

In some embodiments, amorphous polyester polymers are preferred.

Suitable polymers may also be prepared by copolymerization of lactides with monomers such as glycolide, caprolactone, valerolactone and derivatives thereof.

The polyester polymer can have a weight average molecular weight (M_{w}) as measured by the Size Exclusion Chromatography (SEC) test method of from 40,000 Da to 300,000 Da, 70,000 Da to 200,000 Da, from 45,000 Da to 200,000 Da, from 45,000 Da to 150,000 Da, from 50,000 Da to 200,000 Da, from 50,000 Da to 150,000 Da, or even from 50,000 Da to 100,000 Da. The ranges directed to the polyester polymer throughout this document refer to the M_{w} of the polyester polymer after it is produced and prior to use in the hot melt adhesive composition.

The hot melt adhesive composition includes from 10% by weight to 60% by weight, from 10% by weight to 55% by weight, 15% by weight to 60% by weight, from 15% by weight to 55% by weight, or even from 15% by weight to 50% by weight of the polyester polymer.

Useful commercially available PLA based polyester polymers include amorphous PLA resins available under the VERCET trade designation, including e.g. VERCET A-1000, VERCET A-1010X, VERCET A-1020X, VERCET A-1030X, VERCET A-1050 and INGEO 10200D available from NatureWorks LLC (Minnetonka, Minn.) and PLA resins available under the RESOMER trade designation, including e.g. RESOMER RG 504H available from Sigma Aldrich Corporation (St. Louis, Mo.).

Polyester Plasticizer

The hot melt adhesive composition includes a polyester plasticizer. The polyester plasticizer can include more than one polyester plasticizer. The polyester plasticizer can be bio-based. Alternatively, a portion of, or even all the polyester plasticizer can be compostable. The polyester plasticizer can have an oxygenated carbon to total carbon ratio as tested according to the Oxygenated Carbon to Total Carbon Ratio test method of at least 0.38, at least 0.40, at least 0.45, from 0.38 to 0.70, from 0.42 to 0.60, or even from 0.45 to 0.60. The high oxygenated carbon content is thought to improve the compatibility of the polyester plasticizer with the polyester polymer and enable the use of increased amounts of polyester plasticizer without transfer or exudation.

The polyester plasticizer can be derived from monomers selected from the group consisting of 1,2 propanediol, ethylene glycol, succinic acid, isosorbide and combinations thereof. The polyester plasticizer can be derived from monomers selected from the group consisting of 1,2 propanediol, ethylene glycol, succinic acid and combinations thereof.

The polyester plasticizer can have a weight average molecular weight (M_{w}) as measured by the Molecular Weight by Size Exclusion Chromatography (SEC) test method of less than 10,000 Da, less than 7,500 Da, from 1,000 Da to 7,000 Da, or even from 2,000 Da to 6,000 Da.

The hot melt includes greater than 33% by weight, greater than 35% by weight, greater than 40% by weight, greater than 50%, greater than 33% by weight to 85% by weight, greater than 35% by weight to 85% by weight, greater than 40% by weight to 85% by weight, 43% by weight to 85% by weight, 45% by weight to 85% by weight, 48% by weight to 85% by weight, 50% by weight to 85% by weight, 55% by weight to 85% by weight, 60% by weight to 85% by weight, or even 65% by weight to 85% by weight of the polyester plasticizer.

Useful commercially available polyester plasticizers include polyester plasticizers available under the HALLGREEN trade designation including e.g. HALLGREEN R-8010 available from The Hallstar Company (Chicago, Ill.).

HALLGREEN R-8010, per The Hallstar Company, is a 99% USDA Bio certified polymeric ester and completely biodegrades within 42 days in accordance with ASTM D5271 and ISO 14851 and therefore is regarded as completely compostable under aerobic conditions as stated in ASTM D6400 and EN 13432/ISO 17088.

Second Plasticizer

The polyester plasticizer can be used alone or in combination with other plasticizers, including petroleum derived plasticizers. The second plasticizer can include one or more plasticizers.

Useful classes of second plasticizer include, e.g. vegetable oils, animal oils (e.g. glycerol esters of fatty acids) and derivatives thereof, phosphate plasticizers, benzoate plasticizers, other polyesters etc.

The hot melt adhesive can include up to 20% by weight, up to 15% by weight, up to 10% by weight, from 2% by weight to 20% by weight, or even from 5% by weight to 15% by weight of a second plasticizer.

Useful commercially available second plasticizers include benzoate plasticizers available under the BENZOFLEX trade designation including e.g. BENZOFLEX 2088 and BENZOFLEX 9-88 available from Eastman (Kingsport, Tenn.).

Wax

The hot melt adhesive composition can optionally include wax. The wax can include one or more waxes.

Useful classes of wax include, e.g., paraffin waxes, microcrystalline waxes, Fischer-Tropsch waxes, oxidized Fischer-Tropsch waxes, animal waxes, vegetable waxes (e.g. soy wax, rice bran wax, etc.), polyethylene waxes, hydroxy stearamide waxes and combinations thereof. The wax can be functionalized.

In a preferred embodiment, the wax includes polar groups.

The hot melt composition can include from 0% by weight to 15% by weight, from 0% by weight to 10% by weight, from 1% by weight to 10% by weight, or even from 2% by weight to 5% by weight of wax.

Useful commercially available paraffin waxes include e.g., FR-6513 from Citgo Petroleum (Houston, Tex.), SASOLWAX 6705 from Sasol Performance Chemicals (Hamburg, Germany) and PARVAN 1580 from ExxonMobil (Houston, Tex.). Useful commercially available Fischer-Tropsch waxes include SARAWAX SX-70 and SX-80, Fischer-Tropsch waxes from Shell MDS (Bintulu, Malaysia) and SASOLWAX C-80, Fischer-Tropsch wax from Sasol Performance Chemicals (Hamburg, Germany).

Useful waxes include those with higher polarity including e.g. hydroxysteramide waxes such as e.g. PARICIN 220 and PARICIN 285, available from Vertellus (Indianapolis, Ind.).

Tackifying Agents

The hot melt adhesive composition can be free of a tackifying agent, optionally the hot melt adhesive composition can include a tackifying agent.

Useful tackifying agents can be solid or liquid at room temperature. Useful tackifying agents can be selected from the group consisting of bio-based and compostable. Useful tackifying agents can alternatively be derived from non-bio-based materials.

Useful tackifying agents include aromatic resins, terpenes, modified terpenes and hydrogenated versions thereof; natural rosins, modified rosins, rosin esters, and hydrogenated versions thereof; low molecular weight polylactic acid; sucrose and simple sugar derivatives; benzoates; catechol derivatives; and combinations thereof. The tackifying agents can be modified with materials such styrene, phenol, carboxylic acids, anhydrides (e.g., maleic anhydride) and combinations thereof.

Examples of useful natural and modified rosins include gum rosin, wood rosin, tall oil rosin, distilled rosin, hydrogenated rosin, dimerized rosin and polymerized rosin. Examples of useful rosin esters include e.g., glycerol esters of pale wood rosin, glycerol esters of hydrogenated rosin, glycerol esters of polymerized rosin, pentaerythritol esters of natural and modified rosins including pentaerythritol esters of pale wood rosin, pentaerythritol esters of hydrogenated rosin, pentaerythritol esters of tall oil rosin, and phenolic-modified pentaerythritol esters of rosin.

Examples of useful terpene and modified terpenes include those derived from alpha-pinene, beta-pinene, gamma-limonene, dipentene, or mixtures thereof.

Other useful tackifying agents include oligomeric resins derived from other bio-sources such as isosorbide, isomannide, and lignin and alpha methyl styrene resins derived from petroleum feed stocks.

Useful tackifying agents include DERTOPHENE T105, available from DRT (France), KRISTALEX 3100 available from Eastman Chemical Company (Kingsport, Tenn.), MIRAMER SB, a sucrose benzoate tackifier available from Miwon Specialty Chemical Co., Ltd. and KOMOTAC KBD 100L, available from Guangdong KOMO Co. Ltd. (Guangzhou, China).

The tackifying agent can be useful in amounts up to 40% by weight, up to 30% by weight, up to 20% by weight, from 0% by weight to 40% by weight, from 0% by weight to 30% by weight, from 0% by weight to 20% by weight, from 5% by weight to 40% by weight, from 5% by weight to 30% by weight, from 5% by weight to 20% by weight, or even from 5% by weight to 15% by weight.

Second Polymer

The hot melt adhesive composition can include a second polymer different from the polyester polymer.

Useful second polymers include, e.g. styrene block copolymers, acrylic polymers, acrylic block copolymers, ethylene vinyl acetate copolymers (vinyl acetate contents of from 40% by weight to 95% by weight are preferred), polyester polyether copolymers, polyurethanes, polycaprolactone, functionalized versions thereof and combinations thereof.

Useful second polymers can be oligomers. Useful second polymers can be curable (e.g. ultra violet (UV) curable, electron beam curable, etc.). The use of polymers and oligomers that are cured can improve certain properties of the hot melt adhesive composition including e.g. resistance to transfer, heat resistance, shear resistance, etc. Examples of such polymers and oligomers include acrylates, acrylate modified polymers and oligomers (e.g. acrylated polyesters (e.g. acrylated PLA), acrylated polyurethanes, etc.), and high 1,2 vinyl butadiene styrene block copolymers, polycaprolactone, and functionalized versions of the above.

Useful polymers are commercially available under a variety of trade designations including, e.g., the LEVAMELT series of trade designations including LEVAMELT 800, LEVAMELT 456 and LEVAMELT 686 available from Lanxess Corporation (Pittsburgh, Pa.), the ACRESIN series of trade designations including ACRESIN A250 UV and A260 UV from BASF Corporation (Freeport, Tex.), the PEARLBOND series of trade designations including PEARLBOND 501 and PEARLBOND 302 from The Lubrizol Corp. (Wickliffe, Ohio), and the CAPA series of trade designations including CAPA 6250, CAPA 6400, CAPA 6500, and CAPA 6800 from Ingevity (Brussels, Belgium).

Useful additional polymers and oligomers can present at no greater than 30%, no greater than 20% by weight, no greater than 15% by weight, no greater than 10% by weight, from 2% by weight to 30% by weight, from 2% by weight to 20% by weight, from 2% by weight to 15% by weight, or even from 2% by weight to 10% by weight.

Optional Components

The hot melt adhesive composition can optionally include up to 30% by weight of a variety of additional components including, e.g., stabilizers, antioxidants, adhesion promoters, ultraviolet light stabilizers, photo polymerization initiators, rheology modifiers, biocides, corrosion inhibitors, dehydrators, colorants (e.g., pigments and dyes), optical brighteners, fillers, surfactants, flame retardants, additional polymers and oligomers.

Useful antioxidants include, e.g., pentaerythritol tetrakis[3,(3,5-di-tert-butyl-4-hydroxyphenyl)propionate], 2,2′-methylene bis(4-methyl-6-tert-butylphenol), phosphites including, e.g., tris-(p-nonylphenyl)-phosphite (TNPP) and bis(2,4-di-tert-butylphenyl)4,4′-diphenylene-diphosphonite, di-stearyl-3,3′-thiodipropionate (DSTDP), and combinations thereof. Useful antioxidants are commercially available under a variety of trade designations including, e.g., the IRGANOX series of trade designations including, e.g., IRGANOX 1010, IRGANOX 565, and IRGANOX 1076 hindered phenolic antioxidants and IRGAFOS 168 phosphite antioxidant, all of which are available from BASF Corporation (Florham Park, N.J.), and ETHYL 702 4,4′-methylene bis(2,6-di-tert-butylphenol), and the BNX series of trade designations including, e.g., BNX 1010 and BNX 1076 from Mayzo, Inc. (Suwanee, Ga.). When present, the adhesive composition preferably includes from about 0.1% by weight to about 2% by weight antioxidant.

Uses The hot melt adhesive composition can be applied to (i.e. such that it is in direct contact with) or incorporated in a variety of substrates including, e.g., films, release liners, porous substrates, cellulose substrates, sheets (e.g., paper and fiber sheets, paper board, etc.), paper products, woven and nonwoven webs, fibers, tape backings, plastics (e.g. polyethylene terephthalate (PET), polyethylene (HDPE and LDPE), etc.), glass and metals (e.g. aluminum, steel, etc.).

The hot melt adhesive composition is useful on substrates made from a variety of fibers including, e.g., natural cellulose fibers (e.g. wood pulp, cotton, viscose, starch, etc.), silk, PLA, PHA, PBS, PBAT (poly butylene adipate terephthalate) and wool; synthetic fibers such as nylon, rayon, polyesters, acrylics, polypropylenes, polyethylene, polyvinyl chloride, polyurethane, and glass; recycled fibers, and various combinations thereof.

The hot melt adhesive composition is useful on a variety of films including polyethylene, polypropylene, ethylene vinyl acetate, ethylene copolymer, bio-based (e.g. PLA, PHA, PBAT, starch, etc.), partially bio-based (e.g. starch blends).

The hot melt adhesive composition is also useful in a variety of applications and constructions including, e.g., disposable absorbent articles including, e.g., disposable diapers, adult incontinence products, sanitary napkins, medical dressings (e.g., wound care products) bandages, surgical pads, pet training pads (e.g. puppy pads), face masks, and meat-packing products and components of absorbent articles including, e.g., an absorbent element, absorbent cores, impermeable layers (e.g., backsheets), tissue (e.g., wrapping tissue), acquisition layers, labels (e.g., pressure-sensitive adhesive labels), tapes (e.g., repositionable pressure sensitive adhesive tapes), cases, cartons, books and combinations thereof.

The hot melt adhesive composition can be used to make adhesive tapes or alternately to adhere labels to various items (e.g. containers, magazines, etc.). The label/tape can be selected from a variety of materials including paper, non-paper films (e.g. polypropylene (e.g. polypropylene (PP), oriented polypropylene (OP), and biaxially oriented polypropylene (BOPP)), polyethylene, etc.). The container can be metal (e.g. aluminum or steel) or plastic (polyethylene terephthalate (PET), high density polyethylene (HDPE) and polypropylene.

The label can be a spot label i.e. a label that does not extend completely around the container. Alternatively, the label can be a wraparound label i.e. a label that completely wraps around the entire container.

If the label is a wraparound label, it can be roll fed into the applicator. Alternatively, the labels are pre-cut and fed in from a stack. In a wraparound label application method, the label stock is fed into a label station. A pick-up adhesive and a lap glue are then applied to the label, often from the same glue pot. A pick-up adhesive adheres the leading edge of the label to a container. The lap glue then bonds the overlap where the wrap around label overlaps itself. The hot melt adhesive composition of this invention can be both the pick-up adhesive and the lap glue.

The hot melt adhesive compositions of this invention can be used in a wide variety of applications within the disposable absorbent article. The hot melt adhesive compositions can be used as construction adhesives (e.g. used to bond the back sheet to the nonwoven and optionally the absorbent pad), as a positioning adhesives (e.g. to adhere a disposable absorbent article to an undergarment), and for core stabilization (e.g. applying a hot melt composition to the absorbent core to increase the strength of the core).

The hot melt adhesive composition can be used for construction applications. In a typical construction application in the manufacture of a disposable absorbent article, a body fluid impermeable back sheet is bonded to a nonwoven substrate. The hot melt adhesive composition may also be used to bond at least one additional layer or material selected from the group consisting of absorbents, tissues, elastomeric materials, superabsorbent polymers, and combinations thereof. For example, the adhesive can further be used for back sheet lamination i.e. where the body fluid impermeable backsheet typically a film (e.g. polyethylene, polypropylene, ethylene vinyl acetate, ethylene copolymer, bio-based etc.) is bonded to a second nonwoven to improve the feel of the disposable article.

The hot melt adhesive composition can be useful as a positioning adhesive disposed on at least one substrate surface of a disposable absorbent article and can be used to position an absorbent article on a garment such as underwear. Such disposable absorbent articles include, e.g., feminine hygiene articles such as sanitary napkins and panty liners, diapers, disposable garments having a waist opening and leg openings, and adult incontinence articles.

In one construction, the absorbent article (e.g., a feminine hygiene article) includes a garment facing surface and a body facing surface, a topsheet having a garment facing surface and a body facing surface, a backsheet having a garment facing surface and a body facing surface, and an absorbent core disposed between the body facing surface of the backsheet and the garment facing surface of the topsheet.

Various application techniques can be used to apply the composition to a substrate including, e.g., slot coating, spraying including, e.g., spiral spraying and random spraying, screen printing, foaming, engraved roller, extrusion and melt blown application techniques.

The invention will now be described by way of the following examples. All parts, ratios, percentages and amounts stated in the Examples are by weight unless otherwise specified.

### Examples

### Test Procedures

Test procedures used in the examples include the following. All ratios and percentages are by weight unless otherwise indicated. The procedures are conducted at room temperature (i.e., an ambient temperature of from about 20° C. to about 25° C.) unless otherwise specified. The properties set forth for the components used in the compositions are as reported by the manufacturer unless otherwise specified.

### Oxygenated Carbon to Total Carbon Ratio

The ratio was obtained using C-13 Nuclear Magnetic Resonance (NMR) Spectrometer.

In the absence of aromatic or alkene containing species, the summed 13C NMR signals integrals from the polyester plasticizer from 50-200 ppm were ratioed to the total summed integrals assigned to the polyester plasticizer from 0 to 200 ppm to determine the oxygenated carbon content.

If aromatic or alkene structures are present in the polyester plasticizer, the integrals assigned to these structures would be omitted from the 50-200 ppm summation, but still measured as part of the total summed integrals to determine oxygenated carbon content.

### Molecular Weight by Size Exclusion Chromatography (SEC)

The tetrahydrofuran (THF) used for sample preparation and analysis was stabilized with 2,6-Di-tert-butyl-4-methylphenol (BHT). Approximately 0.02 g of each sample was mixed with 10 mL THF on a shaker for several hours at room temperature. All samples yielded clear, colorless solutions which were filtered through a 0.45 μm PTFE filter.

SEC was performed on a Waters Alliance 2695 HPLC with THF as the eluent. The samples were injected at room temperature and separated using three Agilent PLgel Mixed-B columns heated to 40° C. using a column heater, then analyzed by a Waters 2487 dual absorbance detector (at 254 nm and 300 nm), and then a Waters 2414 refractive index (RI) detector at 40° C. The elution time from the RI detector was calibrated to EasiCal polystyrene PS-1 standards (molecular weight=0.58-6,780 kDa). The data was manually integrated using Thermo Scientific™ Chromeleon™ Chromatography Data System (CDS) software.

M_{w} (Weight Average Molecular Weight) is reported in Daltons (Da).

### Viscosity Test Method

Viscosity is determined in accordance with ASTM D3236 entitled, “Standard Test Method for Apparent viscosity of Hot Melt Adhesives and Coating Materials,” (Oct. 31, 1988), using a Brookfield Thermosel viscometer Model RVDV 2, and a number 27 spindle. The results are reported in centipoise (cP) and the test is performed at the specified temperature.

### Peel Test Sample Preparation Method

Each hot melt adhesive composition was applied to substrates with a slot hot melt applicator set to an application temperature of from 110° C. to 150° C., the lamination equipment was set with minimal rewind and unwind tensions so as not to stretch the film.

Each hot melt adhesive composition was applied at a speed of from 190 meters per minute (m/min) to 300 m/min. Both substrates were traveling at the same speed and after the hot melt adhesive was applied the hot melt adhesive coated side was mated with the second substrate with a nip pressure of 5 pounds per linear inch (pli). A comb shim pattern that was 7.62 centimeters (cm) (3 inches (in)) wide, and teeth of the comb shim spaced 1 mm on and 1 mm off, at a coat weight of 6 grams/square meters (g/m²) in the adhesive area was used. The hot melt adhesive composition was applied to the nonwoven and then mated to the film.

First and second substrates were chosen from the following:
a. an oriented polypropylene nonwoven web having a thickness of 0.1 mm (4 mils) and a basis weight of 15.3 grams per square meter (0.45 ounces per square yard)
b. BIOLACE® COZY nonwoven available from Suominen Corporation (Helsinki, Finland) and having a thickness 0.23 mm (9 mil)
c. a nonbreathable layered polyethylene film having a thickness of 0.023 mm (0.9 mil)
d. a breathable polyethylene film having a thickness of 0.023 mm (0.9 mil)

### Dynamic Peel Test Method

Dynamic Peel was determined per ASTM D1876-01 entitled, “Test Method for Determining Peel Resistance of Adhesive (T-Peel Test Method),” with the exception that the test was run at 30.5 centimeters per minute (12 inches per minute) over a period of 10 seconds and seven replicates were run. The samples were run on an IMASS Spec-type test instrument in a 7.62 cm (3 inch) width. The samples were peeled along the machine coating direction in a down web direction. The average peel value over 10 seconds of peeling was recorded and the results were reported in grams. The initial Dynamic Peel value is the value measured 24 hours after the sample is prepared. Seven replicates were tested and the average value were reported in units of grams of force (gf).

### Glass Transition Temperature (T_{g}) Test Method

The glass transition temperature (T_{g}) of the samples was determined on the sample composition using Dynamic Mechanical Analysis (DMA) with a DHR-II instrument at 10 rad/second using the following conditions: the sample was heated to 140° C., held at 140° C. for 1 minute, cooled to −20° C. at 10° C./minute, held at −20° C. for 10 minutes and then heated from −20° C. to 140° C. at a rate of 3° C./minute.

The temperature at the maximum value of Tan Delta on the heating curve was recorded as the T_{g} in ° C.

### Compatibility Test

If the hot melt adhesive composition formed a clear, homogeneous mixture when melted with no phasing upon mixing, it was rated as compatible (C). If the hot melt adhesive composition formed an opaque mixture when melted or formed two different phasing upon mixing, it was rated as not compatible (NC).

### Transfer Test

Film of adhesive was held at room temperature for 24 hours, then a finger was pushed into the film and then removed. If there was transfer of adhesive to the finger, the film was rated: yes, if no transfer, the film was rated: no.

### Exudation Test

Film of adhesive was held at room temperature for 24 hours, then it was evaluated for the formation of exudate on the surface. If there was exudate, the film was rated: yes, if no exudate, the film was rated: no.

Tack Rating—Film touched with finger and then qualitative tack rating given based on how difficult it is to remove finger (i.e. resistance).

Low—No resistance when touched; leathery feel

Moderate—Slight resistance when touched

Moderately High—Resistance when performing test, but finger can be easily removed

High—Some difficulty removing finger from film

Very High—Very difficult to impossible to remove finger from film

### Loop Tack

The Loop Tack was performed according ASTM D 6195-97 Standard Test Methods for Loop Tack.

### Peel Force to Stainless Steel Sample Preparation Method

A laminate is prepared by coating a sample composition onto a treated Mylar film in a 2.54 cm (1.0 in) wide pattern at an add-on weight of 24 g/m² using a slot applicator and then contacting the adhesive strip with a 0.09 mm (3.5 mil) siliconized release film to form a release treated Mylar/adhesive/siliconized release film laminate. Test samples having a length of 23.32 cm (9.18 inches (in)) in the machine direction and 2.54 cm (1.0 in) in the cross-machine direction are then cut from the laminate such that the adhesive pattern is centered in the cross-machine direction of the test sample.

The release layer is then removed and the adhesive is applied to a stainless-steel panel having a length of 20.32 cm (8 in) and a width of 7.62 cm (3 in). In preparing the composite test sample, the adhesive is not pressed down onto the stainless steel.

### Peel Force to Stainless Steel Test Method

Three samples are prepared per the Peel Force to Stainless Steel Sample Preparation Method. Each test sample is placed on a 2-kg mechanical roll-down device and the roller is allowed to pass over the film side of the sample two times, once in the forward direction and once in the backward direction, at a rate of 305 mm/min. A timer is then activated and the sample is placed into the jaws of INSTRON-type peel tester. After one minute, the sample is peeled at a 180-degree angle according to PSTC 101 entitled, “Peel Adhesion of Pressure Sensitive Tape,” and the peel force is recorded. The average peel force of the three samples is reported in gf/25 mm.

### Examples

The adhesive compositions were prepared by combining and mixing the components under nitrogen in the amounts set forth in Tables 2 and 3 in a sigma blade mixer operating at 135-143° C. or in an upright paddle mixer at a temperature of from 135° C.-155° C.

TABLE 1
Oxygenated Carbon Content of Plasticizers

| | Oxygenated Carbon/ |
|---|---|
| | Total Carbon ratio |
| | (as tested by C-13 NMR) |
| HALLGREEN R-8010 | 0.48 |
| BENZOFLEX 2088 (benzoate ester) | 0.30 |
| DYNACOLL 7230 (saturated copolyester) | 0.37 |
| PARAPLEX A-8092 (polymeric ester) | 0.35 |

TABLE 2
Molecular Weight by SEC

| | M_{w} (Daltons) |
|---|---|
| HALLGREEN R8010 | 3,718 |
| VERCET A1000 | 121,000 |
| VERCET A1010X | 72,500 |
| VERCET A1020X | 11,500 |
| VERCET A1030X | 44,900 |

TABLE 3
Hot Melt Adhesive Compositions for Disposable Absorbent Articles

| | Con 1 | Con 2 | Con 3 | Ex. 1 | Ex. 2 | Ex. 3 | Ex. 4 | Ex. 5 | Ex. 6 | Ex. 7 | Ex. 8 |
|---|---|---|---|---|---|---|---|---|---|---|---|
| HALLGREEN R-8010 | | | | 49 | 54 | 60 | 64 | 70 | 75 | 46.5 | 46 |
| DYNACOLL 7230 | 54 | | | | | | | | | | |
| BENZOFLEX 2088 | | 54 | | | | | | | | | |
| PARAPLEX A-8092 | | | 54 | | | | | | | | |
| VERCET A-1010X | 45 | 45 | 45 | 32 | 45 | 39 | 35 | | | 30.4 | 32 |
| VERCET A-1020X | | | | 18.5 | | | | | | 17.6 | 18.5 |
| VERCET A-1000 | | | | | | | | 29 | 24 | | |
| PARICIN 220 | | | | | | | | | | 5 | 3 |
| IRGANOX 1010 | 1.0 | 1.0 | 1.0 | 0.5 | 1.0 | 1.0 | 1.0 | 1.0 | 1.0 | 0.5 | 0.5 |
| Compatibility Test | NC | C | NC | C | C | C | c | C | C | C | C |
| Transfer Test | NT | Yes | NT | No | No | No | No | No | No | No | No |
| Tack Rating | NT | Very | NT | Low | Mod | Mod- | High | High | High | Low | Low |
| | | High | | | | High | | | | | |
| Exudation Test | NT | NT | NT | No | No | No | No | No | No | No | No |
| Viscosity (Visc) @ | NT | NT | NT | 11500 | 13530 | 7850 | 5550 | NT | NT | 4500 | 7250 |
| 225° F. (cP) | | | | | | | | | | | |
| Visc @ 250° F. | NT | NT | NT | NT | NT | NT | NT | 7525 | 4700 | NT | NT |
| (cP) | | | | | | | | | | | |
| T_{g} (° C.) | NT | NT | NT | 19 | 15 | 10 | 6 | 5 | 3 | 20 | 18 |
| Initial Dynamic Peel | NT | NT | NT | 390 | 278 | 267 | 254 | NT | NT | 203 | 224 |
| (gf) - substrates a.) | | | | | | | | | | | |
| and c.) | | | | | | | | | | | |
| 1 week aged Dynamic Peel | NT | NT | NT | 521 | 548 | 456 | 373 | NT | NT | NT | NT |
| (gf) - substrates a.) | | | | | | | | | | | |
| and c.) | | | | | | | | | | | |
| Initial Dynamic Peel | NT | NT | NT | NT | 259 | 267 | 253 | 169 | 151 | NT | NT |
| (gf) - substrates a.) | | | | | | | | | | | |
| and d.) | | | | | | | | | | | |
| Initial Dynamic Peel | NT | NT | NT | 708 | 641 | 617 | 428 | NT | NT | 225 | 327 |
| (gf) - substrates b.) | | | | | | | | | | | |
| and c.) | | | | | | | | | | | |

NT (Not Tested)
Substrates
a.) oriented polypropylene nonwoven web having a thickness of 0.1 mm (4 mils) and a basis weight of 15.3 grams per square meter (0.45 ounces per square yard)
b.) BIOLACE ® COZY nonwoven available from Suominen Corporation (Helsinki, Finland) and having a thickness 0.23 mm (0.9 mil)
c.) nonbreathable layered polyethylene film having a thickness of 0.023 mm (0.9 mil)
d.) breathable polyethylene film having a thickness of 0.023 mm (0.9 mil)

TABLE 4
Hot Melt Pressure Sensitive Adhesive Compositions

| | Ex. 9 | Ex. 10 | Ex. 11 | Ex. 12 | Ex. 13 |
|---|---|---|---|---|---|
| HALLGREEN R-8010 | 79 | 81 | 69 | 64 | 79 |
| BENZOFLEX 2088 | | | 10 | | |
| VERCET A-1010X | | | | 33 | 18 |
| VERCET A-1000 | 20 | 18 | 20 | | |
| PARICIN 220 | | | | 2.0 | 2.0 |
| IRGANOX 1010 | 1.0 | 1.0 | 1.0 | 1.0 | 1.0 |
| Compatibility Test | C | C | C | C | c |
| Tack Rating | Very | Very | Very | High | Very |
| | High | High | High | | High |
| Exudation Test | No | No | No | No | No |
| Visc @ 107° C. | 1352 | 319 | 3688 | 3950 | 3375 |
| (225° F.) (cP) | | | | | |
| T_{g} (° C.) | 0 | −2 | −4 | 7 | 2 |
| 180° Peel (gf/25 mm) | 109 | 47 | 23 | NT | 48 |
| Loop Tack (gf) | 319 | 165 | 233 | NT | 201 |

TABLE 5
Hot Melt Construction and Pressure
Sensitive Adhesive Compositions

| | Ex. 14 | Ex. 15 | Ex. 16 | Ex. 17 | Ex. 18 |
|---|---|---|---|---|---|
| HALLGREEN | 45.5 | 37 | 36 | 46.5 | 50 |
| R-8010 | | | | | |
| VERCET A1010X | 30.5 | 27 | 27 | 35 | 36.5 |
| VERCET A1020X | | 24 | 15 | | |
| PARICIN 220 | 3.5 | 3.5 | 3.5 | 3.5 | 3.5 |
| PEARLBOND 501 | | 8 | 8 | | |
| ACRESIN A260 | | | | 15 | 10 |
| UV | | | | | |
| MIRAMER SB | 20 | | 10 | | |
| IRGANOX 1010 | 0.5 | 0.5 | 0.5 | | |
| Compatibility Test | C | C | C | C | c |
| Tack Rating | Low | Low | Low | Very | Very |
| | | | | High | High |
| Exudation Test | No | No | No | No | No |
| Viscosity @107° C. | 3900 | 16,580 | 16,000 | | |
| (225° F.) (Cp) | | | | | |
| Viscosity @ 121 C. | 1700 | 6475 | 6425 | 8275 | 5600 |
| (250 F.) (Cp) | | | | | |
| T_{g} (° C.) | 22 | 25 | 23 | 11 | 10 |
| 180° Peel | NT | NT | NT | 193 | 167 |
| (gf/25 mm) | | | | | |
| Loop Tack (gf) | NT | NT | NT | 519 | 622 |
| Initial Dynamic | 465 | NT | NT | NT | NT |
| Peel (gf) - substrates | | | | | |
| a.) and b.) | | | | | |

Other embodiments are within the claims.

What is claimed is:

1. A hot melt adhesive composition comprising:
a. from 15% by weight to 60% by weight of a polyester polymer selected from a group consisting of poly lactic acid based polymer, polyhydroxy alkanoate based polymer, and blends thereof and,
b. from greater than 40% by weight to 85% by weight of a polyester plasticizer having an oxygenated carbon to total carbon ratio of at least 0.40 and a weight average molecular weight (M_{w}) as tested by Size Exclusion Chromatography (SEC) of no greater than 10,000 Da.

2. The hot melt adhesive composition of claim 1 having a loop tack of from 50 gf to 2000 gf.

3. The hot melt adhesive composition of claim 1 wherein at least 50% weight of the hot melt adhesive composition comprises components that are compostable.

4. The hot melt adhesive composition of claim 1 wherein at least 70% by weight of the hot melt adhesive composition comprises components that are compostable.

5. The hot melt adhesive composition of claim 1 wherein at least 70% by weight of the hot melt adhesive composition comprising components that are bio-based.

6. The hot melt adhesive composition of claim 1 further comprising from 1% by weight to 10% by weight of a wax.

7. The hot melt adhesive composition of claim 1 further comprising from 5% by weight to 30% by weight of a second polymer selected from the group consisting of polycaprolactone, polyurethane and polyacrylate.

8. The hot melt adhesive composition of claim 1 further comprising a tackifying agent.

9. The hot melt adhesive composition of claim 8 wherein the tackifying agent is selected from bio-based and compostable.

10. The hot melt adhesive composition of claim 1 wherein the polyester plasticizer is compostable.

11. The hot melt adhesive composition of claim 1 wherein the polyester plasticizer is present at from 43% by weight to 85% by weight.

12. The hot melt adhesive composition of claim 1 wherein the polyester plasticizer is present at from 50% by weight to 85% by weight.

13. The hot melt adhesive composition of claim 1 wherein the polyester plasticizer has a weight average molecular weight (M_{w}) as tested by SEC of from 2000 Da to 6000 Da.

14. The hot melt adhesive composition of claim 1 wherein the polyester plasticizer is derived from monomers selected from ethylene glycol, 1,2 propanediol, succinic acid, isosorbide and combinations thereof.

15. The hot melt adhesive composition of claim 1 wherein the polyester polymer is a poly lactic acid based polymer that is amorphous.

16. The hot melt adhesive composition of claim 1 wherein the polyester polymer has a weight average molecular weight (M_{w}) as tested by SEC of from 50,000 Da to 150,000 Da.

17. The hot melt adhesive composition of claim 1, further including a second polymer that is curable.

18. A hot melt adhesive composition comprising:
a. from 15% by weight to 55% by weight of a polyester polymer selected from a group consisting of poly lactic acid based polymer and polyhydroxy alkanoate based polymer, and blends thereof and,
b. from 35% by weight to 85% by weight of a polyester plasticizer having a weight average molecular weight (M_{w}) as tested by SEC of no greater than 10,000 Da.

19. The hot melt adhesive composition of claim 17 wherein the polyester polymer has a weight average molecular weight (M_{w}) as tested by SEC of from 50,000 Da to 150,000 Da.

20. An article selected from the group consisting of a disposable absorbent article, a tape and a label comprising the composition of claim 1.
